(19) United States
(12) Patent Application Publication
(10) Pub. No.: US 2024/0390629 A1
(43) Pub. Date: Nov. 28, 2024

(54) NOSE VENT PLUG, AND NASAL MASK FOR ANESTHESIA

(71) Applicant: BEIJING MINGDASHU MEDICAL TECHNOLOGY CO., LTD., Beijing (CN)

(72) Inventors: Ming TIAN, Beijing (CN); Xialu BAI, Jiangsu (CN)

(73) Assignee: BEIJING MINGDASHU MEDICAL TECHNOLOGY CO., LTD., Beijing (CN)

(21) Appl. No.: 18/688,530

(22) PCT Filed: Jun. 23, 2022
(86) PCT No.: PCT/CN2022/100834

§ 371 Date: Mar. 1, 2024

(30) Foreign Application Priority Data
Sep. 2, 2021 (CN) 202111026861.3

(51) Int. Cl. A61M 16/06

(52) U.S. Cl. CPC A61M 16/0666; A61M 16/0683; A61M 2202/0007; A61M 2202/0241; A61M 2205/0216; A61M 2209/088; A61M 2210/0606; A61M 2210/0618

(57) ABSTRACT

A nose vent plug, and a nasal mask for anesthesia. The nose vent plug comprises a base (1) and two nasal plugs (2) symmetrically provided on the base (1); the inner edges of the two nasal plugs (2) form a gap (3) into which a nasal septum is embedded; each nasal plug (2) comprises an insertion portion (4) and a pressing portion (5); the outer diameter of the pressing portion (5) is larger than that of the insertion portion (4); the bottom end of the pressing portion (5) is integrally formed on the base (1); the insertion portion (4) is integrally formed on the top of the pressing portion (5); an air hole (6) passing through the pressing portion (5) and the base (1) is provided in the insertion portion (4). The insertion portion (4) of the nose vent plug is shorter, and has fewer parts entering a nostril compared with an existing structure, such that human comfort is improved; moreover, the inner-outer contour shape of the nose vent plug is improved, the inner contour of the nose vent plug is used to fit the nasal septum, the pressing portion (5) is used to plug the nostril, and pressing the base (1) against a nasal cavity can strengthen the air tightness of the nasal cavity, such that on the premise of ensuring human comfort, the air tightness between a ventilation structure and nostrils is improved, thereby avoiding air leakage.

### TECHNICAL FIELD

The present application belongs to the technical field of medical instruments, in particular to a nose vent plug and a nasal mask for anesthesia.

### BACKGROUND ART

The nose plays a very important role in the process of oxygen supply, and is an important channel for external oxygen to enter the human body. At the same time, it is also responsible for exhaling carbon dioxide from the body, which is the first link of the human respiratory system.

When the human body is sick, injured or under anesthesia, an external device is often needed to lead oxygen supply or assist breathing. At this time, it is necessary to establish an artificial ventilation structure connected to the nose, such as a nose vent plug and a nasal mask. The existing ventilation structure connected with the nostril often experiences air leakage and causes discomfort to the patient during use, and therefore it is necessary to improve it.

### SUMMARY

The purpose of the present application is to overcome the deficiencies of the prior art, and propose a nose vent plug, which improves the human body experience during use and the air tightness during the ventilation process.

The present application solving its technical problem is realized by taking the following technical solutions:

A nose vent plug comprises a base and two nasal plugs symmetrically provided on the base and inner edges of the two nasal plugs form a gap for embedding a nasal septum, wherein the nasal plug comprises an insertion portion for inserting into a nasal cavity to deliver air flow and a pressing portion for plugging a nostril and supporting wing of nose to prevent the nasal cavity form air leakage, a bottom end of the pressing portion is integrally formed on the base, the insertion portion is integrally formed on a top end of the pressing portion, and an air hole running through the pressing portion and the base is provided in the insertion portion.

An upper portion of the nasal plug is the insertion portion, middle and lower portions of the nasal plug is the pressing portion, whose outer diameter is larger than that of the insertion portion, and a range of an angle α between a tangent line at each point on an outer contour line of the pressing portion and a horizontal line is 20°<α<80°.

A fitting edge is provided on a labial side of the base to fit with a nasolabial fold so that the base can cover the nostril when the inserting portion is fully inserted into the nostril.

Inner edge lines of the two nasal plugs are gradually approached from top to bottom so that the gap is in a convergent shape.

A material of the nose vent plug is medical silica gel or rubber silica gel.

The nose vent plug integrally comprises an external air bag and a filling medium which is filled in the external air bag; and wherein the filling medium is gas, liquid or gel.

A nasal mask for anesthesia comprises a nasal mask main body and a nose vent plug, wherein a connecting pipe is provided in the nasal mask main body, an insertion pipe connected to the connecting pipe is provided on the nasal mask main body, and the nose vent plug is fitted on the insertion pipe through the air hole.

The nasal mask main body integrally is in a shape of a triangular frame hollowed out in the middle and fits cheeks of face around the nose, the nasal mask main body comprises a vent portion and a fixing portion symmetrically connected to both ends of the vent portion, and the connecting pipe provided in the vent portion.

An external connecting pipe for connecting an external air supply device is connected to the connecting pipe.

Fixing straps that can be engaged with each other are respectively provided on the fixing portion.

Advantages and positive effects of the present application are as follows:

1. The insertion portion of the nasal plug is shorter, and has fewer parts entering the nostril during ventilation compared with an existing structure, which can improve the comfort level of the human body.

2. The shape of the inner and outer contours of the nasal plug is improved, and the inner contour of the nasal plug is used to fit the nasal septum of the human body, the insertion portion of the nasal plug is used for ventilation by being inserted into the nasal cavity, and the pressing portion is used to plug the nostril, such that on the premise of ensuring the comfort of the human body, the sealing between the ventilation structure and the nostrils is improved and sealing of the nasal cavity can be strengthened when pressing against the base, avoiding air leakage.

### BRIEF DESCRIPTION OF THE DRAWINGS

FIG. 1 is a three-dimensional view of the overall structure of a nose vent plug;

FIG. 2 is a sectional view of a nose vent plug in Example 1:

FIG. 3 is a sectional view of a nose vent plug in Example 2:

FIG. 4 is a three-dimensional schematic diagram of a nasal mask:

FIG. 5 is a schematic sectional view of a connecting pipe in the nasal mask:

FIG. 6 is a diagram of the usage state of the nasal mask.

Among them, 1, base: 2, nasal plug: 3, gap: 4, insertion portion: 5, pressing portion: 6, air hole; 7, fitting edge: 8, external air bag: 9, filling medium: 10, nasal mask main body: 11, connecting pipe; 12, insertion pipe: 13, vent portion: 14, fixing portion: 15, external connecting pipe: 16, fixing strap.

### DETAILED DESCRIPTION

The present application will be described in further detail in conjunction with accompanying drawing below.

### Example 1

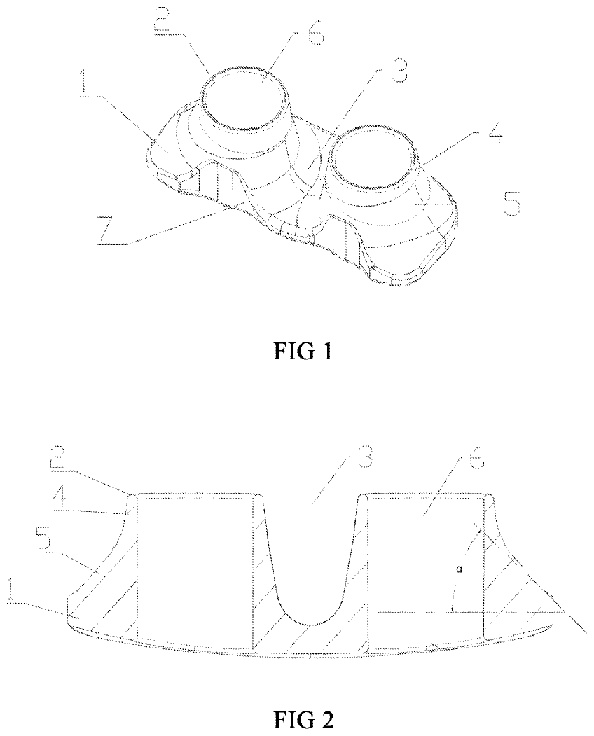

As shown in FIG. 1, a nose vent plug comprises a base 1 and two nasal plugs 2 symmetrically provided on the base 1, and the inner edges of the two nasal plugs 2 form a gap 3 for embedding a nasal septum. The nasal plug 2 comprises an insertion portion 4 and a pressing portion 5. The outer diameter of the pressing portion 5 is larger than that of the insertion portion 4, the bottom end of the pressing portion 5 is integrally formed on the base 1, and the insertion portion 4 is integrally formed on the top of the pressing portion 5. An air hole 6 running through the pressing portion 5 and the base 1 is provided in the insertion portion 4. The insertion portion 4 is used for being inserted into a nasal cavity to deliver air flow, and the pressing portion 5 is used for plugging the nostril to prevent nasal cavity form air leakage.

Specifically, in the present application, the nose vent plug comprises a base 1 and a nasal plug 2. The number of the nasal plug 2 is two, and the two nasal plugs 2 are symmetrically provided on the base 1. When in use, the two nasal plugs 2 can be externally connected to an air supply device, and are respectively inserted into two nostrils of a human. A gap 3 for embedding a nasal septum is formed between the inner edges of the two nasal plugs 2. The outer contour of the existing nasal plug 2 is in the shape of a long drop, and goes deep into the nasal cavity for ventilation. At the same time, the outer contour of the drop-shaped nasal plug 2 fits the inside of the nasal cavity to seal and avoid air leakage during ventilation. However, this method requires the nasal plug 2 to enter the nasal cavity very deeply, and meanwhile requires the outer contour of nasal plug 2 to fit the nasal wing in extremely high extent. In fact, when the nasal plug 2 enters the nasal cavity too deep, it is easy to irritate the mucous membrane in the nasal cavity, causing discomfort such as itching in the nasal cavity, and too much penetration will reduce the empty space in the nasal cavity, leading to high air pressure in the nasal cavity during ventilation, which is prone to cause air leakage and aggravates discomfort. At the same time, it is not possible for the nasal plug 2 to fit the contour of each user's nasal cavity very high, and nor is it possible to make the nasal plug 2 based on the user's nasal cavity before use. Therefore, the shape of this nasal plug 2 and the method of sealing by going deep into the nasal cavity are not suitable.

The applicant believes that in order to improve the comfort of the user, the nasal plug 2 should not penetrate into the nasal cavity too much. At the same time, in order to ensure sealing, the bottom of the nasal plug 2 should be widened and the nasal plug 2 is set in a shape with widening sequentially from top to middle and from middle to bottom. Therefore, in the present application, the nasal plug 2 comprises an insertion portion 4 and a pressing portion 5, wherein an air hole 6 running through the pressing portion 5 and the base 1 is provided in the insertion portion 4 which is responsible for inserting into the nasal cavity for ventilation through the air hole 6, and the pressing portion 5 is used to plug the nostril and prop up the wing of the nose for sealing. When the insertion portion 4 is inserted into the nostril, pressing against the base 1 in the direction of the nasal cavity can strengthen the sealing of the airway in the nasal cavity. In such a way, firstly there is no need for the insertion portion 4 to enter the nasal cavity very deeply, so it will not irritate the mucous membrane in the nasal cavity and cause discomfort such as itching, and will not occupy the space inside the nasal cavity and thus lead to higher air pressure in the nasal cavity during ventilation. Then the pressing portion 5 is used to plug and support the nostril to block the nasal cavity, producing a sealing effect to avoid air leakage. In this way, it can not only avoid the nostril discomfort caused by nasal plug 2, but also ensure the sealing to avoid air leakage.

Further, as shown in FIG. 2, the upper portion of the nasal plug 2 is the insertion portion 4, the middle and lower portions of the nasal plug 2 is the pressing portion 5, whose outer diameter is larger than that of insertion portion 4, and the range of an angle α between the tangent line at each point on the outer contour line of the pressing portion 5 and the horizontal line is 20°<α<80°.

Specifically, since the insertion portion 4 is not suitable for penetrate into the nasal cavity too deeply, in the present application, the upper portion of the nasal plug 2 is the insertion portion 4, that is, the insertion portion 4 accounts for ⅓ of the overall nasal plug 2, and the corresponding middle and lower portions of the nasal plug 2 is the pressing portion 5, that is, the pressing portion 5 accounts for ⅔ of the overall nasal plug 2. The outer contour of the pressing portion 5 is preferably a smooth curve, which is more adaptive when it touches the nostril, and avoids pressing the nostril to cause discomfort. At the same time, the range of the angle α between the tangent line at each point on the outer contour line of the pressing portion 5 and the horizontal line is 200<α<80°, widening from top to bottom gradually, therefore the pressing portion 5 can play a role of propping up the wing of nose, plugging the nostril, and blocking the nasal cavity when facing different sizes of the nostril.

Further, the labial side of the base 1 is provided with a fitting edge 7 to fit with the nasolabial fold so that the base can cover the nostril when the inserting portion is fully inserted into the nostril. The inner edge lines of the two nasal plugs 2 are gradually approached from top to bottom so that the gap 3 is in a convergent shape.

Specifically, when the nose vent plug is in use, the base 1 should cling to the nasolabial fold between the nose and the upper lip, so a fitting edge 7 is provided on the labial side of the base 1, and depressions are provided on the two sides of the middle uplift of the fitting edge 7, being capable of better fitting the nasolabial fold and improving the comfort of use. The nasal septum will be embedded in the gap 3 between the two nasal plugs 2. In order to ensure the stability of wearing and further strengthen the sealing, the inner edge lines of the two nasal plugs 2 should be gradually approached from top to bottom, so that the gap 3 is in a convergent shape, and the nasal septum can be sandwiched therein. And when most of the insertion portion 4 is inserted into the nostril, its base 1 can cover the opening of the nostril, and pressing against the base 1 can strengthen the sealing of the airway in nasal cavity.

The material of the nose vent plug is medical silica gel or rubber silica gel. In this example, the nasal plug 2 is a soft sleeve made of medical silica gel or a soft sleeve made of rubber silica gel, which can ensure comfort when in contact with the nasal cavity, and has a certain degree of elasticity, which can improve the sealing during the contact with the nostril and the nasal septum.

### Example 2

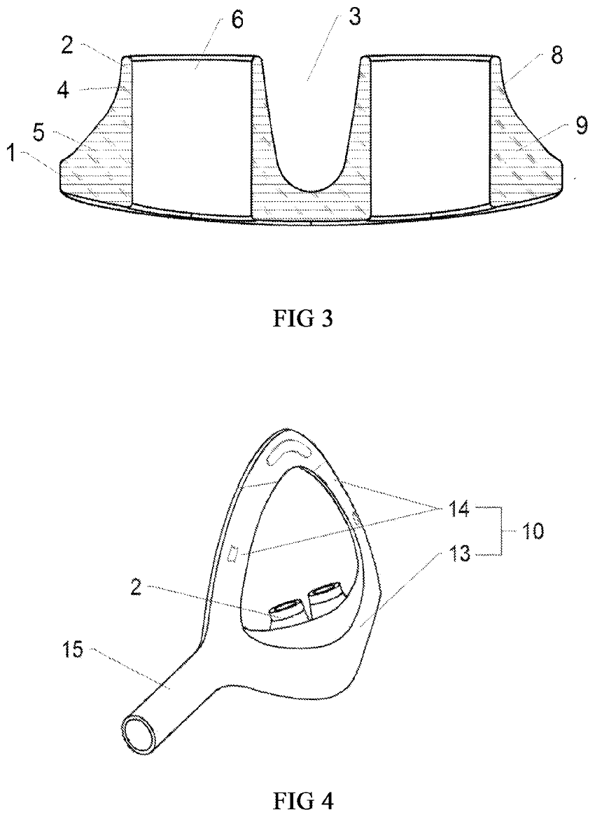

As shown in FIG. 3, the nose vent plug integrally comprises an external air bag 8 and a filling medium 9 filled in the external air bag 8, and the filling medium 9 is gas, liquid or gel.

In this example, the nose vent plug is composed of two parts, the external air bag 8 and the filling medium 9. The external air bag 8 is bulged by filling the filling medium 9 in the external air bag 8, playing a role of ensuring comfort and improving air tightness when in contact with the nasal cavity. This method can make the nose vent plug softer integrally, and compared with example 1, the nose vent plug is more comfortable to wear, however, the air tightness is relatively insufficient.

### Example 3

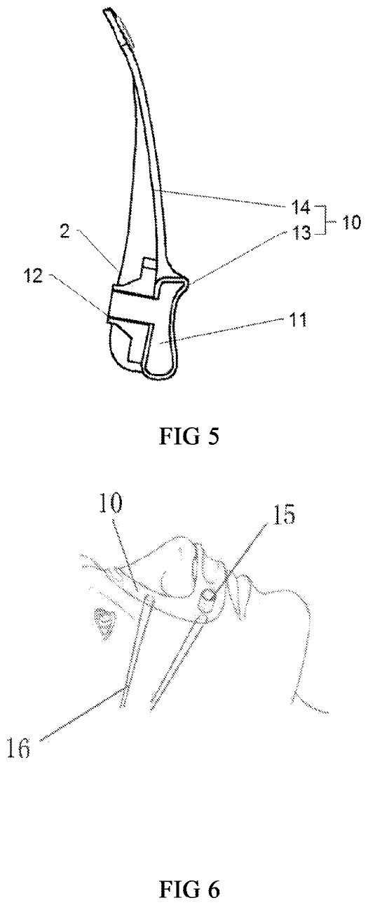

As shown in FIGS. 4, 5 and 6, a nasal mask for anesthesia comprises a nasal mask main body 10 and a nose vent plug. A connecting pipe 11 is provided in the nasal mask main body 10, and an insertion pipe 12 connected to the connecting pipe 11 is provided on the nasal mask main body 10. The nose vent plug is fitted on the insertion pipe 12 through the air hole 6. The nasal mask main body 10 integrally is in the shape of a triangular frame hollowed out in the middle and fits cheeks of face around the nose. The nasal mask main body 10 comprises a vent portion 13 and a fixing portion 14 symmetrically connected to both ends of the vent portion 13. The connecting pipe 11 is provided in the vent portion 13, and an external connecting pipe 15 is connected to the connecting pipe 11. The external connecting pipe 15 is used for connecting an external air supply device.

In this example, a nasal mask for anesthesia with the above-mentioned nose vent plug is proposed. Specifically, the nasal mask comprises a nasal mask main body 10 and a nose vent plug. A connecting pipe 11 is provided in the nasal mask main body 10, and an insertion pipe 12 connected to the connecting pipe 11 is provided on the nasal mask main body 10. The nose vent plug is fitted on the insertion pipe 12 through the air hole 6.

When in use, the nasal mask main body 10 needs to be worn on the nose of the face. Therefore, the nasal mask integrally is in the shape of a triangular frame hollowed out in the middle and fits cheeks of face around the nose. That is, the nasal mask main body 10 comprises a vent portion 13 and a fixing portion 14 symmetrically connected to both ends of the vent portion 13, the triangular frame is formed between the fixing portion 14 and the vent portion 13. When worn, the nose corresponds to the hollow part in the middle of the triangular frame, and the fixing portion 14 fits the face on both sides of the nose. The vent portion 13 is located between the nose and the upper lip, the base 1 of the nose vent plug clings to the nasolabial fold, and the nasal plug 2 is inserted into the nostril to deliver the air flow. Further, an external connecting pipe 15 is connected to the connecting pipe 11, and may be connected to the external air supply device for assisting the human body to breathe.

As shown in FIG. 8, further, fixing straps 16 that can be engaged with each other are respectively provided on the fixing portion 14.

Specifically, fixing straps 16 that can be engaged with each other are respectively provided on the fixing portion 14. When wearing the nasal mask, after the nasal mask main body 10 fits the cheeks of the face around the nose, the fixing straps 16 may be used to fix the nasal mask main body 10, which can avoid the nasal mask from loosening and falling during use. At the same time, the fixing straps 16 tightly binds the nasal mask on the face, which can further make the nasal plug 2 in the ventilation structure closer to and the wing of nose and the nasal septum, improving air tightness.

Further, the applicant conducted a corresponding air tightness comparison test for the existing nasal mask with drop-shaped nasal plug and the above-mentioned nasal mask.

According to body weight, 15 experimenters were divided into three groups: 30-50 kg, 50-70 kg, and 70-100 kg, with 5 people in each group. Each person wore nasal mask A (drop-shaped) and nasal mask B (above-mentioned example 4), and the ventilation end of the nasal mask was connected with a hand-held precision differential pressure gauge. After the differential pressure gauge was set to zero, the experimenter gently pressed the nasal mask with one hand to make it cling to the nostril, held the differential pressure gauge with the other hand and took a deep breath, then exhaled as much as possible. The data on the differential pressure gauge was observed and recorded. The following is a comparison table of experimental data:

TABLE 1
Comparison table of pressure data of 30-50 kg group

| | person | | | | |
|---|---|---|---|---|---|
| type of | 1 | 2 | 3 | 4 | 5 |
| nasal mask | data | | | | |
| A | 16 mmH₂O | 15 mmH₂O | 16 mmH₂O | 16 mmH₂O | 17 mmH₂O |
| B | 40 mmH₂O | 43 mmH₂O | 42 mmH₂O | 43 mmH₂O | 43 mmH₂O |

TABLE 2
Comparison table of pressure data of 50-70 kg group

| | person | | | | |
|---|---|---|---|---|---|
| type of | 6 | 7 | 8 | 9 | 10 |
| nasal mask | data | | | | |
| A | 17 mmH₂O | 16 mmH₂O | 19 mmH₂O | 20 mmH₂O | 18 mmH₂O |
| B | 41 mmH₂O | 43 mmH₂O | 40 mmH₂O | 44 mmH₂O | 44 mmH₂O |

TABLE 3
Comparison table of pressure data of 70-100 kg group

| | person | | | | |
|---|---|---|---|---|---|
| type of | 11 | 12 | 13 | 14 | 15 |
| nasal mask | data | | | | |
| A | 17 mmH₂O | 19 mmH₂O | 20 mmH₂O | 20 mmH₂O | 19 mmH₂O |
| B | 43 mmH₂O | 42 mmH₂O | 45 mmH₂O | 44 mmH₂O | 44 mmH₂O |

According to the experimental data, it can be seen that the pressure data of the experimenter using nasal mask A does not exceed 20mmH₂O, while the pressure data of the experimenter using nasal mask B is above 40mmH₂O. Therefore, it can be seen that the nasal mask using the scheme of the present application has much better air tightness than the existing drop-shaped nasal mask, and can better meet the needs of users.

It should be emphasized that the examples described in the present application are illustrative rather than limiting, and thus the present application includes and is not limited to the examples described in the detailed description. Other embodiments obtained by those skilled in the art based on the technical solution of the present application also fall within the scope of protection of the present application.

1. A nose vent plug, comprising a base (1) and two nasal plugs (2) symmetrically provided on the base (1), and inner edges of the two nasal plugs (2) forming a gap (3) for embedding a nasal septum, wherein the nasal plug (2) comprises an insertion portion (4) for inserting into a nasal cavity to deliver air flow and a pressing portion (5) for pressing against a nostril to block the nasal cavity, a bottom end of the pressing portion (5) is integrally formed on the base (1), the insertion portion (4) is integrally formed on a top end of the pressing portion (5), and an air hole (6) running through the pressing portion (5) and the base (1) is provided in the insertion portion (4).

2. The nose vent plug according to claim 1, wherein an upper portion of the nasal plug (2) is the insertion portion (4), middle and lower portions of the nasal plug (2) is the pressing portion (5) whose outer diameter is larger than that of the insertion portion, and a range of an angle α between a tangent line at each point on an outer contour line of the pressing portion (5) and a horizontal line is 20°<α<80°.

3. The nose vent plug according to claim 1, wherein a fitting edge (7) is provided on a labial side of the base (1) to fit with a nasolabial fold so that the base (1) can cover the nostril when the inserting portion is fully inserted into the nostril.

4. The nose vent plug according to claim 1, wherein inner edge lines of the two nasal plugs (2) are gradually approached from top to bottom so that the gap (3) is in a convergent shape.

5. The nose vent plug according to claim 1, wherein a material of the nose vent plug is medical silica gel or rubber silica gel.

6. The nose vent plug according to claim 1, wherein the nose vent plug integrally comprises: an external air bag (8), and a filling medium (9) which is filled in the external air bag (8); and wherein the filling medium (9) is gas, liquid or gel.

7. A nasal mask for anesthesia, comprising a nasal mask main body (10) and the nose vent plug of claim 1, wherein a connecting pipe (11) is provided in the nasal mask main body (10), an insertion pipe (12) connected to the connecting pipe (11) is provided on the nasal mask main body (10), and the nose vent plug is fitted on the insertion pipe (12) through the air hole (6).

8. The nasal mask for anesthesia according to claim 7, wherein the nasal mask main body (10) integrally is in a shape of a triangular frame hollowed out in the middle and fits cheeks of face around the nose, the nasal mask main body (10) comprises a vent portion (13) and a fixing portion (14) symmetrically connected to both ends of the vent portion (13), and the connecting pipe (11) is provided in the vent portion (13).

9. The nasal mask for anesthesia according to claim 8, wherein an external connecting pipe (15) for connecting an external air supply device is connected to the connecting pipe (11).

10. The nasal mask for anesthesia according to claim 8, wherein fixing straps (16) that can be engaged with each other are respectively provided on the fixing portion (14).
